(19) United States
(12) Patent Application Publication
(10) Pub. No.: US 2005/0251011 A1
(43) Pub. Date: Nov. 10, 2005

(54) Clinical trial image and data processing system

(76) Inventors: Gudrun Zahlmann, Neumarkt (DE); Siegfried Schneider, Erlangen (DE); Markus Schmidt, Nuernberg (DE)

Correspondence Address: Alexander J. Burke;Intellectual Property Department, 5th Floor, 170 Wood Avenue South, Iselin, NJ, 08830, US

(21) Appl. No.: 10/945,700

(22) Filed: Sep. 21, 2004

Related U.S. Application Data
(60) Provisional application No. 60/564,509, filed on Apr. 22, 2004.
(60) Provisional application No. 60/564,510, filed on Apr. 22, 2004.

(51) Int. Cl. A61B 8/00

(52) U.S. Cl. 600/407

(57) ABSTRACT

A communication system connects clinical trial participants, managers, sponsors and associated systems and provides imaging-specific functions that enhance imaging systems. A patient clinical image data processing system includes an acquisition interface for acquiring patient medical image representative data from one or more sources. A processor automatically removes patient specific information from patient medical image representative data to provide patient non-specific medical image representative data and stores the patient non-specific medical image representative data. A communication interface communicates, via a network, the patient non-specific medical image representative data to a remote location.

This is a non-provisional application of provisional applications Ser. No. 60/564,509 by M. Schmidt et al. filed Apr. 22, 2004 and Ser. No. 60/564,510 by M. Schmidt et al. filed Apr. 22, 2004.

### FIELD OF THE INVENTION

This invention concerns a system and user interface supporting image based clinical trials involving trial sponsors and other participants.

### BACKGROUND INFORMATION

Existing systems typically acquire image information in clinical trial processes through manual action. A site with an imaging modality (CT, MR, X-ray, ultra-sound etc.) uses imaging procedures and parameters that seem to produce images in a more or less reproducible way according to each user's expertise with the imaging modality. Images in digital format are stored on a CD-ROM and sent together with a case report form (CRF) detailing clinical trial information to a sponsor, for example. The sponsor employs a service company that removes patient identification information (i.e. that renders image information anonymous). The sponsor collates and stores the processed image and associated data in a database, cleans and verifies the data and secures the database. The collated data is analyzed to produce trial results. Existing systems employ largely manual processes in performing the described tasks and these processes offer limited efficiency and data comparability.

Existing image-based clinical trials typically acquire images using imaging systems at care providers and imaging centers and communicate acquired images to trial sponsors. A number of problems arise in this process including the fact that, the different imaging systems involved use incompatible technology. Specifically, the imaging systems involved are provided by different vendors, employ different data formats as well as different user interfaces and quality control processes, for example. Further, the image acquisition units of known trial systems typically serve the needs of physicians in clinical settings and have numerous superfluous features and parameters and do not address the needs of clinical trials in a controlled environment. Known systems typically employ various different methods for rendering, clinical and image data, patient non-specific and anonymous at various locations including at a trial site, in-between site and sponsor etc. Various methods and levels of quality control are employed by the different systems and the systems may lack audit trail management capability. Known systems also lack integration between imaging related processes in a clinical trial and the other processes and also lack defined, common interfaces supporting communication between trial participants (sites, sponsors, etc.).

Known systems typically manually perform clinical trial image information management steps that are not comprehensively standardized. This, in conjunction with the lack of interface technology and processes results in an increased burden in performing image-based clinical trials that is compounded by incompatible system results. Further, existing solutions are not readily adapted for use in new trials and environments. A system according to invention principles addresses these deficiencies and problems.

### SUMMARY OF THE INVENTION

A system and customizable user interface consistently renders clinical and image trial data patient non-specific (anonymous), employs a common data Format for interchange of trial clinical data and images (e.g. using DICOM or CDISC) and also provides process quality control and audit trail monitoring. A patient clinical image data processing system includes an acquisition interface for acquiring patient medical image representative data from one or more sources. A processor automatically removes patient specific information from patient medical image representative data to provide patient non-specific medical image representative data and stores the patient non-specific medical image representative data. A communication interface communicates, via a network, the patient non-specific medical image representative data to a remote location.

### BRIEF DESCRIPTION OF THE DRAWING

FIG. 1 illustrates a communication and data exchange system connecting participants in clinical trial involving image data, according to invention principles.

FIG. 2 shows a system supporting a clinical trial involving image data, according to invention principles.

FIG. 3 shows a flowchart of a process employed by the system of FIG. 2 for supporting a clinical trial involving image data, according to invention principles.

### DETAILED DESCRIPTION OF INVENTION

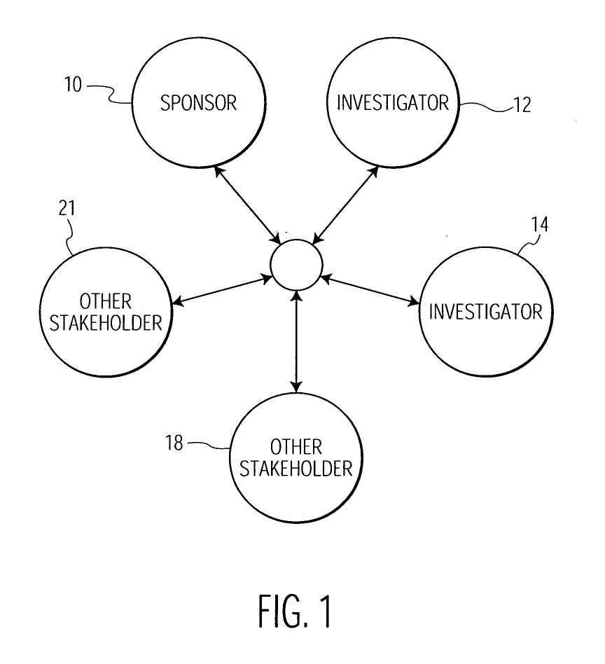

FIG. 1 illustrates a communication and data exchange system connecting participants in a clinical trial involving image data. The FIG. 1 system comprises a continuous infrastructure that connects clinical trial participants and systems. A clinical trial typically involves a sponsor 10, one or more investigators 12 and 14, and other stakeholders 18 and 21 such as trial subjects, regulatory authorities and supervisory authorities. A trial subject is recruited based on qualification to be a test subject in a trial. Further, a trial may be conducted concurrently (or during separate different time period) at multiple remotely located sites.

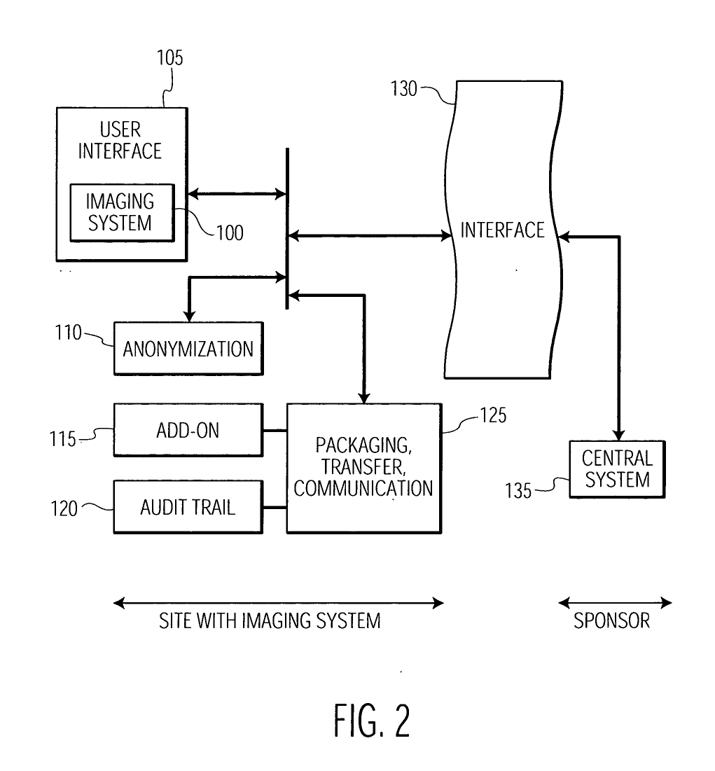

FIG. 2 shows a system supporting a clinical trial involving image data and facilitating data exchange and communication between the participants shown in FIG. 1. The system consistently processes clinical and image trial data to render the data, patient non-specific (anonymous) and employs a consistent, common data Format for interchange of trial clinical data and images (e.g. using DICOM or CDISC). A common user interface employed by imaging devices and other devices used in a trial, provides a standard image layout and set of user functions (that may be restricted and simplified by a trial sponsor, for example) for use by trial participants. A quality control processor ensures standard operating procedures are employed in image acquisition, for example and consistent pricing information is set for use by trial participants. An audit processor maintains a record of data interchange for audit trail purposes.

The system provides cost effective, efficient, clinical trial management with improved quality and security at trial sites and comparability of trial results from different trial sites through use of increased automation. The automation is applied in image acquisition and data transfer as well as in rendering patient medical data anonymous. The system is also advantageously integrated with existing workflow task sequence operation at trial sites and provides workflow management for clinical trial management. The system supports multi-site clinical trials as well as trials at just one site. Also system functions may be used for other purposes including supporting training, education, patient identification and site identification, for example.

The clinical trial system of FIG. 2 supports image associated clinical trials by providing a comprehensive infrastructure and process framework. A communication system (as illustrated in FIG. 1) connects clinical trial participants, managers, sponsors and associated systems and enables data exchange and communication. The imaging-specific functions (user friendly user interface, audit trail generation, patient record anonymity implementation, etc.) are used in conjunction with image processing functions (image acquisition, image management, etc.) in a preferred embodiment. In an alternative embodiment the imaging specific functions may be provided separately and independently. The system addresses the problems presented by running clinical trials at multiple remote heterogeneous sites and ensures consistent and common use of a processor for rendering trial associated image data anonymous (non-patient specific).

The system provides an interface enabling acquisition of images, data, etc. from a site by a sponsor site with a reduced processing burden. The system facilitates addition of more sites, in response to sponsor request, without significant additional burden. The system enables trial sites to share their imaging capabilities with different sponsors in a straightforward manner. The interface is able to require that additional information is generated including audit trail identification information, quality control parameters, etc. In particular, clinical trials involving medical images may require infrastructure supporting transport of images (involving transport of high volume data) with functions including maintaining an audit trail, secure messaging and interfacing to a variety of different systems. These systems include imaging modalities (computerized tomography (CT), magnetic resonance (MR), X-ray, ultra-sound and other devices) as well as interfacing to a Radiology Information Systems (RIS) and a Picture Archiving Computerized System (PACS), for example. The clinical trial interface also includes acquisition units for acquiring and processing clinical trial data including data from imaging devices trials. The interface processing involves management of images, blinded read processes for image and data interpretation including single and double blind test processing as well as anonymization and authorization management through the use of security protocols (e.g. SSL) together with electronic signature management, for example.

An executable application comprises code or machine readable instruction for implementing predetermined functions including those of an operating system, healthcare information system or other information processing system, for example, in response user command or input. A processor as used herein is a device and/or set of machine-readable instructions for performing tasks. As used herein, a processor comprises any one or combination of, hardware, firmware, and/or software. A processor acts upon information by manipulating, analyzing, modifying, converting or transmitting information for use by an executable procedure or an information device, and/or by routing the information to an output device. A processor may use or comprise the capabilities of a controller or microprocessor, for example. A display processor or generator is a known element comprising electronic circuitry or software or a combination of both for generating display images or portions thereof. A user interface comprises one or more display images enabling user interaction with a processor or other device. An object comprises a grouping of data, executable instructions or a combination of both or an executable procedure.

The clinical trial system of FIG. 2 includes modules 100-135 that provide a comprehensive clinical trial infrastructure and process framework. In the system, imaging modality 100, located at a trial site, acquires medical images. User interface (UI) 105 supports operation of modality 100 and enables user selection of a normal mode or an image-based clinical trial mode with a single click of a data entry device (mouse, keyboard, touchscreen, voice activation unit etc.). User interface 105 is configurable (customizable) to offer a restricted choice to a user of both, image layout and available functions and associated buttons, in an image-based clinical trial application setting. This simplification ensures a degree of commonality of user interface display between trial sites and both reduces training burden and minimizes likelihood of user error. User interface 105 facilitates use of imaging system 100 for clinical studies (not just for conventional clinical routine) and ensures use of a consistent set of parameters enabling comparability of the images within a clinical study. User interface 105 initiates generation of data representing a composite display image that includes image elements for initiating different functions supporting a clinical trial in response to user activation. Such functions include generating (by using processor 110) and displaying patient non-specific medical image representative data from patient specific medical image representative data. Audit processor 120 supports accessing and displaying data comprising a record identifying user access to patient specific or non-specific medical image representative data.

Processor 110 renders patient medical data (including medical image data) anonymous, verifies identity and security of users and information sources and supports identification of users and information sources at a particular trial site. Unit 115 provides additional functions including maintaining pricing information to support billing. Unit 115 also includes a security manager (implementing continuous security that is related to a role selected by a user or user profile information associated with user identification) and a quality control processor. The quality control processor ensures quality of clinical images and adherence to operating procedures, for example. For this purpose, the quality control processor in unit 115 monitors image quality, the degree of compliance with predetermined standards and the degree of compliance with predetermined procedures. Further, the quality control processor, in response to a determined deficiency at least one of, initiates generation of an alert message to a user and initiates remedial action to correct the determined deficiency.

Audit processor 120 tracks events and allows compliance with country specific laws and requirements. Such laws include, for example, 21 CFR part 11, a specific regulation that deals with the use of electronic records and signatures in manufacturing processes regulated by the United States Food and Drug Administration (FDA). Audit processor 120 includes logging and tracking capabilities that monitors and records access and modification of data by data and user and system. Processor 120 records information identifying data and users that have accessed or attempted to access patient clinical trial and other medical information. Data collator 125 accumulates, aggregates and formats data including images and other clinical data rendered anonymous by processor 110 as well as audit trail information and other information in a format that is predetermined by the system. Unit 125 further includes a bidirectional communication function for transferring and communicating processed data via interface 130 to a central system 135 supporting clinical trial management by a trial sponsor, for example.

Bidirectional interface 130 enables a trial sponsor employing central system 135 to select characteristics and requirements that determine operation of quality control functions within the FIG. 2 system and also to determine operational functions of user interface 105 of imaging system 100. Interface 130 also enables a trial sponsor to configure interface 130 to receive data in a selected predetermined format. Interface 130 is a flexible, common interface that connects an image source (e.g., imaging system 100) in an image based clinical trial (such as performed by a healthcare provider) and a sponsor or trial manager. Central system 135 includes a clinical trial PACS (Picture Archiving Computerized System) and incorporates a Radiology Information System (RIS) and collates and integrates images from systems of different vendors. Interface 130 provides a well defined interface that performs image acquisition and image management and advantageously allows healthcare providers to establish sites for image based clinical trials and facilitates connection between trial sites and a sponsor. Configurable common interface 130, between central system 135 of the sponsor and one or more trial sites, reduces the problems involved in supporting data exchange between multiple trial sites with disparate, incompatible information systems.

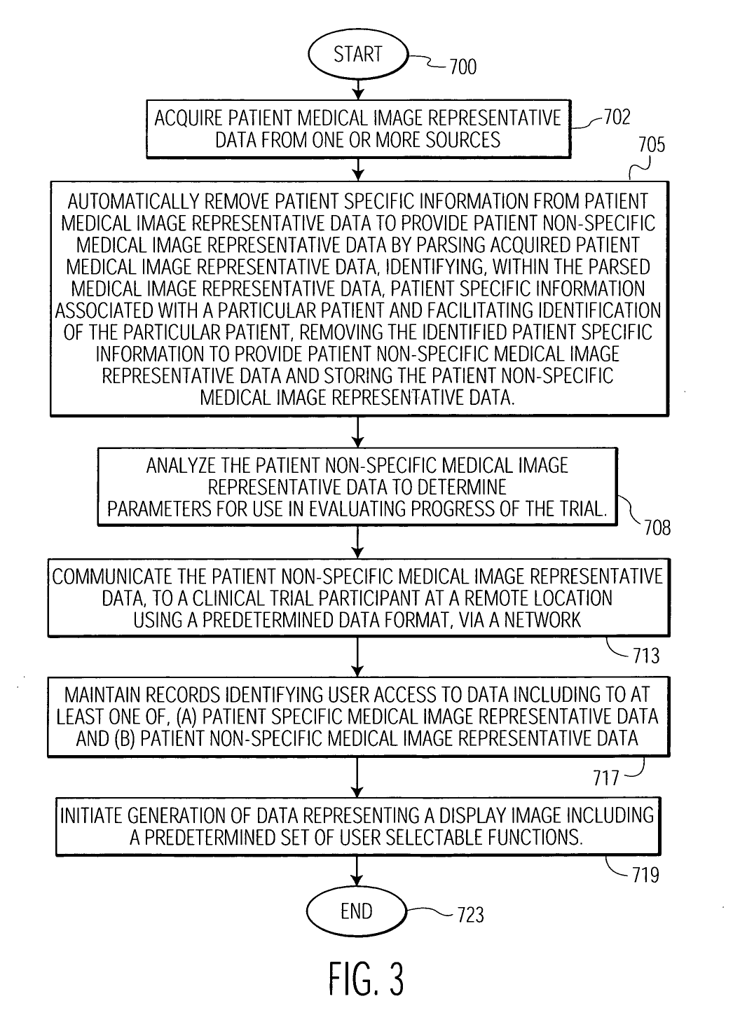

FIG. 3 shows a flowchart of a process employed by the system of FIG. 2 for supporting a clinical trial involving image data. The process of image based clinical trials employs a pre-defined detailed trial protocol that describes the procedure of data acquisition from trial subjects (answering questions, measuring parameters like blood pressure and acquiring images of specific regions, etc.). Eligible patients are identified and trial investigators follow the protocol and acquire images in addition to other information about individual patients. The acquired data is processed (by verification, anonymization, quality control, adding of audit trail information, etc.) at a site where it is compiled. The processed data is transferred to a central system where it is collated. In the process in step 702, following the start at step 700, an acquisition interface in unit 105 (FIG. 2) acquires patient medical image representative data from one or more sources such as imaging system 100.

Anonymization unit 110 in step 705 automatically parses acquired patient medical image representative data to identify, within the parsed medical image representative data, patient specific information associated with a particular patient that facilitates identification of the particular patient. Unit 110 removes the identified patient specific information to provide patient non-specific medical image representative data. Specifically, unit 110 removes a text string associated with a patient name, a patient identifier, patient address, patient contact information, patient medical insurance information or data associated with a patient healthcare provider organization or physician, for example Unit 110 stores the resulting patient non-specific medical image representative data in memory in unit 110 or in another embodiment in another unit or a remote unit via a network. Unit 115 in step 708 analyzes the patient non-specific medical image representative data to select and determine parameters for use in evaluating progress of the trial. Unit 115 selects and determines the parameters for individual images to facilitate consistent image comparison in a trial (e.g., between multiple trial sites).

Interface 130 in step 713 communicates the patient non-specific medical image representative data via a network to a trial participant at remote central system 135 or another remote location using a predetermined data format. A trial participant may be a trial investigator, a person acting as a trial sponsor, a trial sponsor organization or an independent trial auditor, for example. Audit unit 120 in step 717 maintains audit trail records identifying user access to data including patient specific medical image representative data and patient non-specific medical image representative data. The audit trail records include, identification information associated with a user attempting to access the data, identification information of data accessed or identification information of a device source of a request to access the data. In step 719 user interface 105 initiates generation of data representing a display image including a predetermined set of user selectable functions. The predetermined set of user selectable functions are common to user interface display images available at multiple different trial locations and provide consistency of display image layout between multiple different trial locations. The user interface 105 display image includes a predetermined set of parameters derived for individual images associated with the patient non-specific medical image to enable consistent comparison of images in a trial. The process of FIG. 3 terminates at step 723.

The system embodiments include different network architectures. One embodiment uses a centralized database together with applications for analysis and management that are hosted by a sponsor in central system 135, for example, or an outsourcing company. Other participants use client applications that are connected with the central system 135 to access the system functions. In another embodiment, a peer-to-peer architecture is employed in which data and applications are distributed across participating systems. A further embodiment employs a mixed architecture including a central management system and distributed data storage.

The system and processes presented in FIGS. 1-3 are not exclusive. Other systems and processes may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. Further, any of the functions provided by the systems and process of FIGS. 1-3 may be implemented in hardware, software or a combination of both. The system is usable in other research environments not just healthcare environments. In a further embodiment, the system integrates image based clinical trial processes and non-image based clinical trial processes (using Electronic Data Capture (EDC) software and data management systems, for example). The system may be offered to users as an ASP (Application Service Provider) hosted service whereby database and analysis applications are offered with pricing models such as pay-per-image, pay-per-patient, pay-per-study, etc. Further, the data and the applications may be provided either by complete or partial outsourcing. The system may also be offered for sale together with services such as implementation or training, for example. In an alternative business model, image-related functions (enhancing existing systems within their environments) may be offered for sale to users separately. Alternatively, in a further embodiment, reimbursement may be made dependent on the particular use to which the trial data is to be put.

1. A patient clinical image data processing system, comprising:
an acquisition interface for acquiring patient medical image representative data from one or more sources; and
a processor for automatically removing patient specific information from patient medical image representative data to provide patient non-specific medical image representative data and storing said patient non-specific medical image representative data; and
a communication interface for communicating via a network said patient non-specific medical image representative data to a remote location.

2. A system according to claim 1, wherein
said processor removes patient specific information associated with a particular patient comprising at least one of, (a) a text string associated with a patient name, (b) a patient identifier, (c) patient address, (d) patient contact information, (e) patient medical insurance information and (f) data associated with a patient healthcare provider organization or physician.

3. A system according to claim 1, including
an audit processor for maintaining records identifying user access to data including to at least one of, (a) patient specific medical image representative data and (b) patient non-specific medical image representative data,
said records including at least one of, (i) identification information associated with a user attempting to access said data, (ii) identification information of data accessed and (iii) identification information of a device source of a request to access said data.

4. A system according to claim 1, wherein
said patient non-specific medical image representative data is for use in a clinical trial and
said communication interface communicates said patient non-specific medical image representative data to a trial participant, at said remote location, comprising at least one of, (a) a trial investigator, (b) a person acting as a trial sponsor, (c) a trial sponsor organization and (d) an independent trial auditor.

5. A system according to claim 1, including
a user interface for initiating generation of data representing a display image including at least one of, (a) said patient non-specific medical image representative data and (b) data comprising a record identifying user access to patient specific or non-specific medical image representative data.

6. A system according to claim 1, including
a user interface for initiating generation of data representing a composite display image including image elements responsive to user activation for initiating a function comprising at least one of, (a) generating said patient non-specific medical image representative data from patient specific medical image representative data and (b) accessing data comprising a record identifying user access to patient specific or non-specific medical image representative data.

7. A system according to claim 1, wherein
said patient non-specific medical image representative data is for use in a clinical trial and including
a data analyzer for analyzing said patient non-specific medical image representative data to determine parameters for use in evaluating progress of said trial.

8. A system according to claim 7, wherein
said data analyzer determines said parameters consistently for individual images to facilitate image comparison in said trial.

9. A system according to claim 1, wherein
said processor removes patient specific information in accordance with predetermined regulatory requirements.

10. A patient clinical image data processing system, comprising:
an acquisition interface for acquiring patient medical image representative data from one or more sources; and
a processor for automatically removing patient specific information from patient medical image representative data to provide patient non-specific medical image representative data and storing said patient non-specific medical image representative data;
a communication interface for communicating via a network said patient non-specific medical image representative data to a clinical trial participant at a remote location using a predetermined data format; and
a user interface for initiating generation of data representing a display image including a predetermined set of user selectable functions.

11. A system according to claim 10, wherein
said predetermined set of user selectable functions are common to user interface display images available at plurality of different trial locations and provide consistency of display image layout between said plurality of different trial locations.

12. A system according to claim 10, wherein
said display image includes a predetermined set of parameters derived for individual images associated with said patient non-specific medical image to enable consistent comparison of images in said trial.

13. A system according to claim 10, including
a process manager for determining at least one of, (a) image quality and (b) degree of compliance with predetermined standards and (c) degree of compliance with predetermined procedures.

14. A system according to claim 13, wherein
said process manager, in response to a determined deficiency at least one of, (a) initiates generation of an alert message to a user and (b) initiates remedial action to correct said determined deficiency.

15. A method for processing patient clinical image data, comprising the activities of:
acquiring patient medical image representative data from one or more sources; and
automatically removing patient specific information from patient medical image representative data to provide patient non-specific medical image representative data and storing said patient non-specific medical image representative data; and
communicating via a network said patient non-specific medical image representative data to a remote location.

16. A method for processing patient clinical image data, comprising the activities of:
acquiring patient medical image representative data from one or more sources; and
automatically removing patient specific information from patient medical image representative data to provide patient non-specific medical image representative data and storing said patient non-specific medical image representative data;
communicating via a network said patient non-specific medical image representative data to a clinical trial participant at a remote location using a predetermined data format; and
initiating generation of data representing a display image including a predetermined set of user selectable functions.

17. A patient clinical image data processing system, comprising:
an acquisition interface for acquiring patient medical image representative data from one or more sources; and
a processor for automatically,
parsing acquired patient medical image representative data,
identifying, within said parsed medical image representative data, patient specific information associated with a particular patient and facilitating identification of said particular patient,
removing said identified patient specific information to provide patient non-specific medical image representative data and
storing said patient non-specific medical image representative data.

18. A system according to claim 17, wherein
said processor identifies and removes patient specific information associated with a particular patient comprising at least one of, (a) a text string associated with a patient name, (b) a patient identifier, (c) patient address, (d) patient contact information, (e) patient medical insurance information and (f) data associated with a patient healthcare provider organization or physician.

19. A system according to claim 17, including
an audit processor for maintaining records identifying user access to data including to at least one of, (a) patient specific medical image representative data and (b) patient non-specific medical image representative data,
said records including at least one of, (i) identification information associated with a user attempting to access said data, (ii) identification information of data accessed and (iii) identification information of a device source of a request to access said data.

20. A system according to claim 17, wherein
said patient non-specific medical image representative data is for use in a clinical trial and including
a communication interface for communicating via a network said patient non-specific medical image representative data to a trial participant.

21. A system according to claim 20, wherein
said trial participant includes at least one of, (a) a trial investigator, (b) a person acting as a trial sponsor, (c) a trial sponsor organization and (d) an independent trial auditor.

22. A system according to claim 17, including
a user interface for initiating generation of data representing a display image including at least one of, (a) said patient non-specific medical image representative data and (b) data comprising a record identifying user access to patient specific or non-specific medical image representative data.

23. A system according to claim 17, including
a user interface for initiating generation of data representing a composite display image including image elements responsive to user activation for initiating a function comprising at least one of, (a) generating said patient non-specific medical image representative data from patient specific medical image representative data and (b) accessing data comprising a record identifying user access to patient specific or non-specific medical image representative data.

24. A system according to claim 17, wherein
said patient non-specific medical image representative data is for use in a clinical trial and including
a data analyzer for analyzing said patient non-specific medical image representative data to determine parameters for use in evaluating progress of said trial.

25. A system according to claim 24, wherein
said data analyzer determines said parameters consistently for individual images to facilitate image comparison in said trial.

26. A system according to claim 17, wherein
said patient non-specific medical image representative data is for use in a clinical trial and including
a user interface for initiating generation of data representing a display image including a predetermined set of parameters derived for individual images associated with said patient non-specific medical image to enable consistent comparison of images in said trial.

27. A system according to claim 17, including
a process manager for determining at least one of, (a) image quality and (b) degree of compliance with predetermined standards and (c) degree of compliance with predetermined procedures.

28. A system according to claim 27, wherein
said process manager, in response to a determined deficiency at least one of, (a) initiates generation of an alert message to a user and (b) initiates remedial action to correct said determined deficiency

29. A method for processing patient clinical image data, comprising the activities of:
acquiring patient medical image representative data from one or more sources;
automatically parsing acquired patient medical image representative data;
identifying, within said parsed medical image representative data, patient specific information associated with a particular patient and facilitating identification of said particular patient;
removing said identified patient specific information to provide patient non-specific medical image representative data; and
storing said patient non-specific medical image representative data.
